(19) United States
(12) Patent Application Publication
(10) Pub. No.: US 2018/0140372 A1
(43) Pub. Date: May 24, 2018

(54) SURGICAL MICROSCOPE APPARATUS

(71) Applicant: MITAKA KOHKI CO., LTD., Tokyo (JP)

(72) Inventor: Katsuyuki NAKAMURA, Tokyo (JP)

(73) Assignee: MITAKA KOHKI CO., LTD., Tokyo (JP)

(21) Appl. No.: 15/358,646

(22) Filed: Nov. 22, 2016

(51) Int. Cl. A61B 90/25; A61B 46/10

(52) U.S. Cl. CPC A61B 90/25; A61B 2090/506; A61B 46/10

(57) ABSTRACT

A surgical microscope apparatus has a discharge pump unit that is attached to a rear arm of a parallel linkage mechanism and is exposed to an outside. The discharge pump unit, therefore, is easy to remove from a stand body when repairing or maintaining the same. Namely, the repair or maintenance of the discharge pump unit is easy to carry out. The discharge pump unit is not present inside a horizontal arm, and therefore, never increases the weight of the horizontal arm.

### BACKGROUND OF THE INVENTION

### 1. Field of the Invention

The present invention relates to a surgical microscope apparatus covered with a drape when used.

### 2. Description of Related Art

A surgical microscope apparatus used for neurosurgical operations and the like supports a surgical microscope in a suspended state. The apparatus includes a stand body set on the floor, a vertically extending parallel linkage mechanism supported with a shaft on the stand body, a horizontal arm horizontally extending from an upper end of the parallel linkage mechanism, and the surgical microscope suspended from a front end of the horizontal arm.

The surgical microscope apparatus is covered with a drape that keeps at least the surgical microscope and horizontal arm in an aseptic condition. As explained in Japanese Patent Publication No. 2988980, the drape, if sags, obstructs the vision of a doctor or an assistant, and therefore, a discharge pump unit is installed in the horizontal arm, to draw air from the inside of the drape so that the drape is tightly attracted to the surgical microscope apparatus.

### SUMMARY OF THE INVENTION

The related art incorporates the discharge pump unit within the horizontal arm in advance. This configuration makes it difficult to maintain or repair the discharge pump unit. In addition, the incorporated discharge pump unit increases the weight of the horizontal arm, and therefore, a counterweight arranged in the surgical microscope apparatus for realizing a total weight balance must be heavy and bulky.

In consideration of the problems of the related art, the present invention provides a surgical microscope apparatus capable of realizing an easy maintenance of the discharge pump unit and reducing the weight of the horizontal arm.

According to a first aspect of the present invention, the surgical microscope apparatus has a stand body set on one of the floor and ceiling, a parallel linkage mechanism having parallel front and rear arms and installed on the stand body so that the parallel linkage mechanism may tilt in front and rear directions around a rotary shaft arranged at an intermediate part of the front arm, a forwardly extending horizontal arm whose base end forms an upper part of the parallel linkage mechanism, a surgical microscope supported at a front end of the horizontal arm, and a drape covering at least the surgical microscope and horizontal arm. The horizontal arm has a hollow structure and is provided with an intake port at the front end thereof and a connection port at the base end thereof. The surgical microscope apparatus also includes a discharge pump unit attached to one of the rear and front arms of the parallel linkage mechanism and provided with a suction pipe at an upper part of the discharge pump unit, the suction pipe being inserted into the connection port, and a discharge pipe at a lower part of the discharge pump unit, the discharge pipe having an outlet port at a lower end of the discharge pipe.

According to a second aspect of the present invention, the discharge pump unit is attached to the rear arm of the parallel linkage mechanism.

According to a third aspect of the present invention, a part around the connection port at the base end of the horizontal arm serves as a removable cover.

### BRIEF DESCRIPTION OF THE DRAWINGS

FIG. 1 is a side view illustrating a surgical microscope apparatus according to an embodiment of the present invention;

FIG. 2 is a perspective view illustrating a discharge pump unit attached to a parallel linkage mechanism of the apparatus; and

FIG. 3 is a perspective view illustrating a front end area of a horizontal arm of the apparatus.

### DESCRIPTION OF PREFERRED EMBODIMENTS

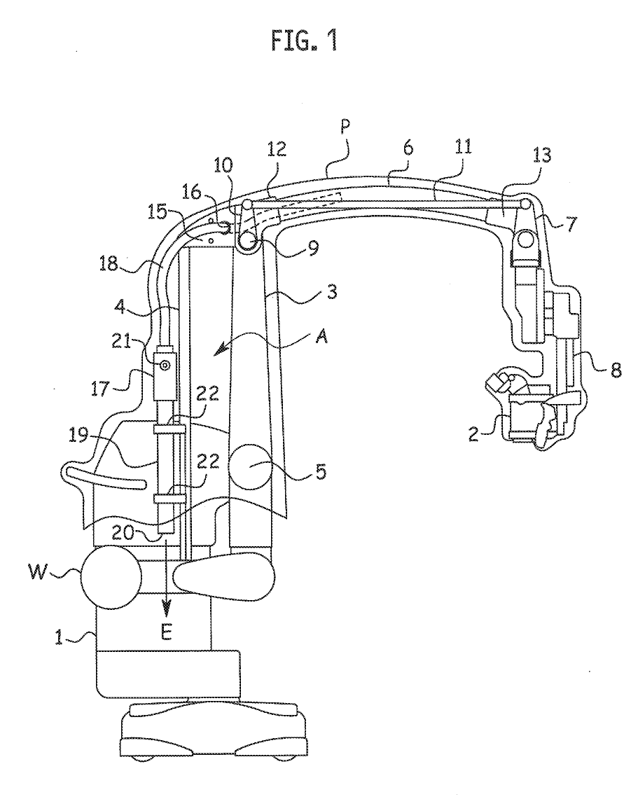

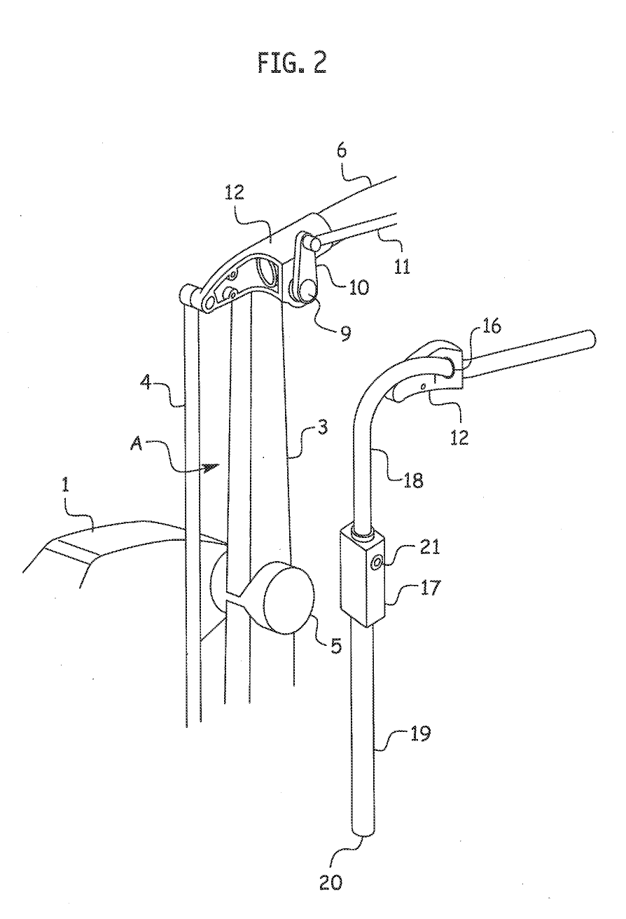

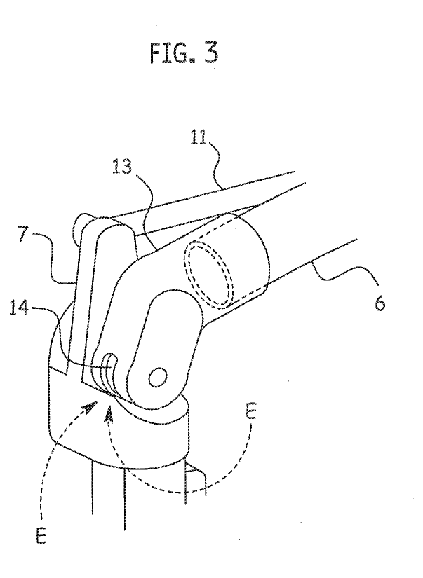

A surgical microscope apparatus according to an embodiment of the present invention will be explained in detail with reference to FIGS. 1 to 3.

The surgical microscope apparatus includes a stand body 1 that is set in an operation room and is used to suspend a surgical microscope 2. The stand body 1 supports a parallel linkage mechanism A having a front arm 3 and a rear arm 4. The parallel linkage mechanism A is tiltable in front and rear directions around a rotary shaft 5 arranged at an intermediate part of the front arm 4.

Arranged at a lower part of the parallel linkage mechanism A is a counterweight W. Forwardly extended from an upper part of the parallel linkage mechanism A is a horizontal arm 6. A front end of the horizontal arm 6 supports through a shaft a lower end of a front arm 7. The lower end of the front arm 7 supports a suspension arm 8 that supports the surgical microscope 2.

The horizontal arm 6 has a fulcrum 9 on the front arm 3. The fulcrum 9 supports a lower end of a base arm 10. The base arm 10 is always kept vertical with a second parallel linkage (not illustrated) incorporated in the front arm 3. An upper end of the base arm 10 is connected through a sub-arm 11 to an upper end of the front arm 7, thereby forming a third parallel linkage. The third parallel linkage always keeps the front arm 7 vertical even when the horizontal arm 6 serving as a horizontal link of the third parallel linkage is turned in up and down directions around the fulcrum 9.

The horizontal arm 6 is made of metal, has a cylindrical shape, and is upwardly curved. The reason why the horizontal arm 6 is upwardly curved is to secure a work space under the same. Ends of the hollow horizontal arm 6 are provided with hollow members 12 and 13 made of cast metal. The horizontal arm 6 including the hollow members 12 and 13 as structural parts is thoroughly hollow. A rear part of the hollow member 12 behind the front arm 3 serves as a part (a horizontal link) of the parallel linkage mechanism A. Namely, the horizontal arm 6 is interlocked with the parallel linkage mechanism A.

The hollow member 13 at the front end of the horizontal arm 6 has an opening, i.e., an intake port 14. A part of the rear hollow member 12 behind the base arm 10 is discrete to serve as a removable cover 15. The cover 15 has an opening serving as a connection port 16. The intake port 14 and connection port 16 are end openings of an air passageway formed through the horizontal arm 6.

Over the stand body 1, a drape P is laid to cover required parts including the surgical microscope 2 and horizontal arm 6 and keep these parts aseptic. If the part of the drape P covering the horizontal arm 6 and surgical microscope 2 sags, it will block the view fields of doctors and assistants. To prevent this, the drape P must tightly be attached to the horizontal arm 6 and surgical microscope 2.

For this, the hollow horizontal arm 6 is used as an air passageway to draw air E through the intake port 14 from the inside of the drape P. The air suction is carried out by a discharge pump unit 17. The discharge pump unit 17 is installed before the drape P is laid.

The discharge pump unit 17 incorporates a pump and has an elongated shape. Arranged at an upper part of the discharge pump unit 17 is a flexible suction pipe 18 and arranged at a lower part thereof is a straight discharge pipe 19. A lower end of the discharge pipe 19 is an outlet port 20. The discharge pump unit 17 has a switch 21 and an electric cord (not illustrated) connected to a power source of the stand body 1.

An installation of the discharge pump unit 17 will be explained. First, the cover 15 is removed from the hollow member 12 of the horizontal arm 6. The suction pipe 18 of the discharge pump unit 17 is inserted into the connection port 16 of the cover 15. At the same time, the suction pipe 18 is inserted into an end of the horizontal arm 6 that is visible inside the hollow member 12, and the cover 15 is fitted to the hollow member 12.

When the cover 15 is fitted to the hollow member 12, the discharge pipe 19 of the discharge pump unit 17 is attached to the rear arm 4 with two clamps 22. The discharge pump unit 17 attached to the rear arm 4 extends along the rear arm 4 and the suction pipe 18 inserted into the horizontal arm 6 extends therein by a predetermined length from the connection port 16. In this way, the attaching of the discharge pump unit 17 to the parallel linkage mechanism A is carried out simultaneously with the fitting of the cover 15 to the horizontal arm 6. Namely, the insertion of the suction pipe 18 into the horizontal arm 6 is easy to achieve.

After the attaching of the discharge pump unit 17, the drape P is laid over the surgical microscope apparatus including the discharge pump unit 17. After the drape P is laid, the switch 21 of the discharge pump unit 17 is pressed to reduce pressure in the horizontal arm 6. Air E within the drape P is drawn from the front intake port 14, passes through the horizontal arm 6 and discharge pump unit 17, and is discharged from the outlet port 20 at the lower end of the discharge pipe 19 to the outside of the drape P.

The horizontal arm 6 is a horizontal link of the third parallel linkage that keeps the front arm 7 vertical. Such a horizontal arm 6 acts as the air suction passageway and is provided with the intake port 14 at a front end of the air suction passageway. Namely, the air E in the drape P is drawn from the front end of the horizontal arm 6 through the inlet 14, and therefore, the drape P around the surgical microscope 2 and horizontal arm 6 is surely and tightly attracted thereto, thereby preventing the drape P from blocking the view fields of persons positioned around the surgical microscope apparatus.

The discharge pump unit 17 attached to the rear arm 4 is exposed to the outside, and therefore, is easily removed from the stand body 1 when repairing or maintaining the discharge pump unit 17, thereby making the repair or maintenance work easy.

The discharge pump unit 17 is not present inside the horizontal arm 6. Instead, it is attached to the rear arm 4, which is opposite to the surgical microscope 2 with respect to the rotary shaft 5 of the parallel linkage mechanism A. As a result, the weight of the discharge pump unit 17 tends to cancel the weight of the surgical microscope 2. Namely, the counterweight W for realizing a total weight balance of the stand body 1 can be lightened by the weight of the discharge pump unit 17.

Although the embodiment mentioned above attaches the discharge pump unit 17 to the rear arm 4, it may be attached to the front arm 3.

According to the first aspect of the present invention, the discharge pump unit is attached to one of the rear and front arms of the parallel linkage mechanism and is exposed to the outside. This aspect makes the discharge pump unit be easily removed from the stand body when repairing or maintaining the discharge pump unit, thereby making the repair or maintenance work easy. This aspect does not arrange the discharge pump unit inside the horizontal arm, and therefore, never increases the weight of the horizontal arm.

The second aspect of the present invention attaches the discharge pump unit to the rear arm of the parallel linkage mechanism, so that the weight of the discharge pump unit may cancel the weight of the surgical microscope. This results in reducing the size of the counterweight for achieving a total weight balance of the stand body.

According to the third aspect of the present invention, the part around the connection port at the proximal portion of the horizontal arm is formed as the removable cover. The suction pipe of the discharge pump unit is inserted into the connection port of the cover in advance, and when the cover is fitted to the proximal portion of the horizontal arm, the discharge pump unit is attached to the parallel linkage mechanism. This aspect makes the insertion work of the suction pipe into the horizontal arm easy.

1. A surgical microscope apparatus comprising:
a stand body;
a parallel linkage mechanism having parallel front and rear arms and installed on the stand body so that the parallel linkage mechanism tilts in front and rear directions around a rotary shaft arranged at an intermediate part of the front arm;
a forwardly extending horizontal arm having a base end that defines an upper part of the parallel linkage mechanism;
a surgical microscope supported at a front end of the horizontal arm, the horizontal arm having a hollow structure and being provided with an intake port at the front end of the horizontal arm and a connection port at the base end of the horizontal arm;
a drape covering at least the surgical microscope and horizontal arm; and
a discharge pump unit removably attached to one of the rear and front arms of the parallel linkage mechanism and provided with a suction pipe at an upper part of the discharge pump unit, the suction pipe being inserted into the connection port, and a discharge pipe at a lower part of the discharge pump unit, the discharge pipe having an outlet port at a lower end of the discharge pipe.

2. The surgical microscope apparatus of claim 1, wherein the discharge pump unit is attached to the rear arm of the parallel linkage mechanism.

3. The surgical microscope apparatus of claim 1, wherein a removable cover conceals the connection port at the base end of the horizontal arm.

4. The surgical microscope apparatus of claim 1, further comprising a counterweight, wherein the weight of the counterweight is based on the weight of the discharge pump.

5. The surgical microscope apparatus of claim 1, wherein the discharge pump is exposed to the outside.
